(19) United States
(12) Patent Application Publication
(10) Pub. No.: US 2025/0302082 A1
(43) Pub. Date: Oct. 2, 2025

(54) METHODS FOR PROMOTING HEALTHY CATCH-UP-GROWTH

(71) Applicant: ABBOTT LABORATORIES, Abbott Park, IL (US)

(72) Inventors: José Maria López Pedrosa, Cenes de la Vega (ES); Mª Pilar Bueno Vargas, Granada (ES); Jorge García Martínez, Armilla (ES); Richardo Rueda Cabrera, Granada (ES)

(21) Appl. No.: 18/866,697

(22) PCT Filed: Jul. 26, 2023
(86) PCT No.: PCT/US2023/028667

§ 371 Date: Nov. 18, 2024

(30) Foreign Application Priority Data
Jul. 29, 2022 (EP) 22382730.4

(51) Int. Cl. A23L 33/00

(52) U.S. Cl. CPC A23L 33/40; A23L 33/30

(57) ABSTRACT

A method of promoting healthy catch-up growth in a pediatric individual comprises administering a nutritional composition with an exosome-enriched product comprising intact bovine milk-derived exosomes to the pediatric individual during a period of weight gain. A method also provides promoting healthy catch-up growth in an underweight individual by administering an exosome-enriched product comprising intact bovine milk-derived exosomes to the individual.

### FIELD OF THE INVENTION

The present invention relates to methods for promoting healthy catch-up growth in a pediatric individual with an exosome-enriched product comprising intact bovine milk-derived exosomes during a period of weight gain, as well as methods for promoting healthy catch-up growth in underweight individuals with an exosome-enriched product comprising intact bovine milk-derived exosomes.

### BACKGROUND OF THE INVENTION

Millions of children around the world suffer from malnutrition. Wasting and stunted growth are the most prevalent forms of malnutrition in children. Wasting, namely being too thin for height, often indicates recent and severe weight loss, which can also be persistent for a prolonged period of time. Stunted growth, or stunting, describes the condition in which children are considered too short for their age. Wasting and stunting commonly occur when a person has not had food of adequate quality and quantity required for a normal, healthy diet, or when a person has suffered from frequent or prolonged illness. Wasting and stunting in children specifically is associated with a higher risk of death if not properly treated.

Usually, once the cause of malnutrition is determined, and proper treatment administered, (for example, an oral nutritional supplementation) catch-up growth naturally happens. While catch-up growth includes weight gain, weight gain itself does not necessarily indicate healthy growth. Weight gain can be especially unhealthy when it occurs at a faster than normal rate. Previous epidemiological studies have indicated that infants and children who have recovered from malnutrition and show catch-up growth are found to have a disproportionate increase in body fat compared to lean body mass. This greater replenishment of body fat, or catch-up fat, can increase long-term health risks such as the development of metabolic syndrome, obesity, higher risk of cardiovascular disease, and insulin resistance during adulthood due to the disproportionate gain of fat mass compared to lean body mass during the catch-up growth period.

Catch-up fat can occur in infants and children during a growing period, but catch-up fat can also affect adolescents and adults recovering weight after a period of weight loss. An increase in a rate of fat gain relative to lean mass in individuals who are recovering weight after a period of weight loss, has been referred to as fat or weight overshooting.

In view of the above, there is an urgent need to develop new and accessible technologies that improve catch-up growth in underweight children, particularly growth as it relates to increasing lean body mass as opposed to fat mass.

Accordingly, methods of promoting healthy catch-up growth during periods of weight gain in children are desirable.

### SUMMARY OF THE INVENTION

It is therefore an object of the invention to provide a convenient means for improving growth in an individual.

In one embodiment, the invention is directed to a method of promoting healthy catch-up growth in a pediatric individual, comprising administering a nutritional composition with an exosome-enriched product comprising intact bovine milk-derived exosomes to the pediatric individual during a period of weight gain.

In another embodiment, the invention is directed to a method for promoting healthy catch-up growth in an underweight individual, comprising administering an exosome-enriched product comprising intact bovine milk-derived exosomes to the individual.

The nutritional compositions comprising an exosome-enriched product comprising intact bovine milk-derived exosomes and methods of promoting healthy catch-up growth, are advantageous in that they provide a convenient, therapeutic strategy for improving the gain of healthy weight in pediatric individuals. These and additional objects and advantages of the invention will be more fully apparent in view of the following detailed description.

### BRIEF DESCRIPTION OF THE DRAWINGS

The drawings are illustrative of certain embodiments of the invention and are exemplary in nature and are not intended to limit the invention defined by the claims, wherein:

FIG. 1A illustrates lean body mass in an animal model group with a non-restricted diet and a group with a restricted diet, after a restriction period, as described in Example 2.

FIG. 1B illustrates fat mass in an animal model group with a non-restricted diet and a group with a restricted diet, after a restriction period, as described in Example 2.

FIG. 2 illustrates growth rate of an animal model group with a restricted diet, after a refeeding period following the restriction period, including a group fed a base diet, and a group fed a base diet including an exosome-enriched product comprising intact bovine milk-derived exosomes, as described in Example 2.

FIG. 3 illustrates body weight of an animal model group with a restricted diet, after a refeeding period following the restriction period, including a group fed a base diet, and a group fed a base diet including an exosome-enriched product comprising intact bovine milk-derived exosomes, as described in Example 2.

FIG. 4A illustrates body composition in terms of lean body mass compared to body weight of an animal model group with a restricted diet, after a refeeding period following the restriction period, including a group fed a base diet, and a group fed a base diet including an exosome-enriched product comprising intact bovine milk-derived exosomes, as described in Example 2.

FIG. 4B illustrates body composition in terms of fat mass compared to body weight of an animal model group with a restricted diet, after a refeeding period following the restriction period, including a group fed a base diet and a group fed a base diet including an exosome-enriched product comprising intact bovine milk-derived exosomes, as described in Example 2.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

Bovine milk contains exosomes which are bilayer membrane vesicles of approximately 20-200 nm in diameter. Exosomes contain several bioactive agents, including, but not limited to enzymatic and non-enzymatic proteins, nucleic acids, and lipids. Bovine milk exosomes can be isolated from a milk whey fraction or from other dairy streams. Procedures for isolating bovine milk exosomes include both physical and chemical methods known in the art. These isolation methods yield a fraction enriched in bovine milk exosomes that can undergo further processing, such as freeze-drying or spray-drying, to produce a powder of bovine milk-derived exosomes for desired end-use applications.

The terms “bovine milk-derived exosomes”, and “exosome-enriched product” as used herein, unless otherwise specified, refer to an enriched bovine milk-derived product comprising bovine milk exosome fractions that have been isolated from a milk whey fraction and enriched in accordance with procedures known in the art as discussed above, and these terms are used interchangeably. The exosomes have been substantially separated from other bovine milk components such as lipids, cells, and debris, and are concentrated in an amount higher than that found in bovine milk. The exosomes are small, extracellular vesicles and account for a minor percentage of milk's total content. In specific embodiments, the exosome-enriched product is provided in a liquid form or a powdered form and also contains co-isolated milk solids.

In a specific embodiment of the invention, the bovine milk-derived exosomes comprise at least 0.001 wt % exosomes. In another specific embodiment, the enriched product of bovine milk-derived exosomes comprises at least about 0.01, 1, 5, 10, 15, 20, 25, 30, 35, 40, 45, or 50 wt % exosomes. In additional specific embodiments of the invention, the enriched product of bovine milk-derived exosomes comprise at least 10 wt % exosomes. In further embodiments, the exosome-enriched product comprises at least about 108 exosomes per gram of the exosome-enriched product as measured by a nanotracking procedure. Briefly, nanoparticle tracking analysis (NTA) can be used to determine exosome diameter and concentration. The principle of NTA is based on the characteristic movement of nanosized particles in solution according to the Brownian motion. The trajectory of the particles in a defined volume is recorded by a camera that is used to capture the scatter light upon illumination of the particles with a laser. The Stokes-Einstein equation is used to determine the size of each tracked particle. In addition to particle size, this technique also allows determination of particle concentration.

In a more specific embodiment, the exosome-enriched product employed in the present invention comprises from about 108 to about 1014 exosomes per gram of the exosome-enriched product. In yet a more specific embodiment, the exosome-enriched product comprises from about 109 to about 1013 exosomes per gram of the exosome-enriched product. In another specific embodiment, the exosome-enriched product contains at least about a three-fold increase in the number of exosomes, as compared to a raw whey-containing bovine milk fraction. In a more specific embodiment, the exosome-enriched product contains a 3-fold to 50-fold increase in the number of exosomes, as compared to a raw whey-containing bovine milk fraction, for example cheese whey.

The term “intact bovine milk-derived exosomes” as used herein, refers to exosomes in which the vesicle membrane is not ruptured and/or otherwise degraded and the endogenous cargo, i.e., bioactive agents, therapeutics (e.g. miRNA), and/or other biomolecules which are inherently present in a bovine milk-derived exosome, are retained therein in active form. In a specific embodiment, at least about 50 wt % of exosomes in the exosome-enriched product are intact. In another specific embodiment, at least about 55, 60, 65, 70, 75, 80, 85, 90, or 95 wt % of the exosomes in the exosome-enriched product are intact.

The term “pediatric” as used herein, unless otherwise specified, refers to an individual at or under the age of about 19 years old, which normally corresponds from the time of birth to when an adult identity and behavior are accepted.

The term “adolescent” as used herein, unless otherwise specified, refers to an individual between the ages of about 10 years to 19 years old, which normally corresponds with the onset of physiologically normal puberty and ends when an adult identity and behavior are accepted.

The term “adult” as used herein, unless otherwise specified, refers to an individual above the age of about 19 years old.

The term “catch-up growth” as used herein, unless otherwise specified, refers to body growth that occurs at a rate greater than normal for age. Clinically, catch-up growth has been observed following a period of growth inhibition due to a variety of causes, including but not limited to, malnutrition and glucocorticoid excess.

Another type of catch-up growth occurs when an individual is progressing through a period of weight gain, particularly after a period of restriction.

The term “catch-up fat” as used herein, unless otherwise specified, refers to a disproportionately higher rate in the recovery of body fat mass compared to lean body mass during a catch-up growth period. Catch-up fat may also be referred to as a “catch-up fat phenotype” referring to when children who recover from a wasting condition typically gain a higher quantity of fat mass at the expense of a lower recovery of lean body mass, regardless of whether an ideal weight is achieved. The catch-up fat phenotype has been associated with an impaired glucose metabolism as well as a higher fat mass content in adulthood.

The catch-up fat phenotype does not only happen in infants and children undergoing catch-up growth, but can also occur in adolescents and adults recovering from a weight loss process, leading to increased visceral adipose tissue.

The terms “moderate malnutrition” and/or “moderately malnourished” as used herein, unless otherwise specified, refers to a weight-for-age of a pediatric individual with a z-score (or standard score) between about −3 and −2 below the median of the World Health Organization (WHO) child growth standards. In a specific embodiment of the invention, the pediatric individual is moderately malnourished.

In another embodiment of the methods of the invention, the pediatric individual is malnourished, with a z-score below the median of WHO child growth standards. In yet another embodiment of the invention, the pediatric individual is severely malnourished with a z-score more than about −3 below the median WHO child growth standards.

The term “weight gain” as used herein, unless otherwise specified, refers to an increase in overall body mass.

The term “growth rate” as used herein, unless otherwise specified, refers to the rate at which overall body mass is increased, or the rate of weight gain.

The term “period of weight gain” as used herein, unless otherwise specified, refers to a refeeding period or recovery period or a period of weight recovery as a designated period of time in which an increase in body mass is desired, following a period of growth restriction.

The term “catch-up diet” as used herein, unless otherwise specified, refers to a diet consumed during a period of weight gain aimed to cause catch-up growth.

The term “underweight” as used herein, unless otherwise specified, refers to a body mass of an individual that is under the average body mass for that individual's representative population, or underweight as determined by calculating an individual's body mass index (BMI). A BMI calculation is based on an individual's height and weight for adults. For pediatric individuals, calculating BMI depends on several factors including age and gender in addition to height and weight. Generally speaking, a BMI below 18.5 is considered underweight.

The term “lean body mass” as used herein, unless otherwise specified, refers to the portion of body mass made up of organs, bones, muscles, skin and fluids that are not fat.

The term “fat mass” as used herein, unless otherwise specified, refers to the portion of body mass made up of fat or adipose tissue.

The term “severe weight loss” as used herein, unless otherwise specified, refers to a decrease in body weight in either a rapid amount or rapid pace in comparison to average or acceptably normal weight loss weights or amounts. What is considered severe weight loss may depend on a variety of factors such as age, height, starting weight, biological sex, genetics, etc.

The term “administration” as used herein, unless otherwise specified, refers to enteral administration, or to administration involving the esophagus, stomach, and small and large intestines (i.e., the gastrointestinal tract). Examples of enteral administration include oral, including sublingual, and tube feeding.

The term “chronic” or “chronically” as used herein, unless otherwise specified, refers to a duration of nutritional intervention of about 5 consecutive days or more.

As indicated above, the present invention provides methods for promoting healthy catch-up growth in a pediatric individual. The present inventors have surprisingly discovered the unexpected result that catch-up growth is improved by administering an exosome-enriched product comprising intact bovine milk-derived exosomes to a pediatric individual after a period of weight restriction.

Avoiding catch-up fat is desired in order to promote healthy catch-up growth. In embodiments of the invention, healthy catch-up growth is achieved by at least one of enhancing development of lean body mass, reducing development of catch-up fat (or reducing fat mass accumulation), increasing growth rate, increasing weight, or combinations of at least two or more thereof, during a period of weight gain.

In one embodiment, the invention is directed to a method of promoting healthy catch-up growth in a pediatric individual, comprising administering a nutritional composition comprising an exosome-enriched product comprising intact bovine milk-derived exosomes to the pediatric individual during a period of weight gain.

In another embodiment, the invention is directed to a method for promoting healthy catch-up growth in an underweight individual, comprising administering an exosome-enriched product comprising intact bovine milk-derived exosomes. In a specific embodiment, the exosome-enriched product comprising intact bovine milk-derived exosomes promotes lean body mass development during a catch-up growth period without increasing fat mass over normal gain levels that are typically acquired in normal growth in pediatric individuals, thereby promoting healthy catch-up growth. Healthy catch-up growth promotes lean body mass versus a higher replacement of fat stores to help acquire a healthier growth pattern.

In a specific embodiment of the invention, the dosage of the exosome-enriched product comprising the intact bovine milk-derived exosomes is from about 0.01 to about 30 g. More specifically, the dosage of the exosome-enriched product comprising the intact bovine milk-derived exosomes may be from about 0.1 to about 30 g, from about 0.1 to about 15 g, or from about 1 to about 15 g. The exosome-enriched product comprising the intact bovine milk-derived exosomes can be administered to an individual at any of the above dosages from about 1 to about 6 times per day or per week, or from about 1 to about 5 times per day or per week, or from about 1 to about 4 times per day or per week, or from about 1 to about 3 times per day or per week.

In a specific embodiment, the exosome-enriched product comprising intact bovine milk-derived exosomes can be administered to an individual chronically during a designated period, such as during a period of weight gain. By way of example, the dosage of the exosome-enriched product comprising the intact bovine milk-derived exosomes may be from about 0.01 to about 30 g/day, from about 0.1 to about 30 g/day, from about 0.1 to about 15 g/day, or from about 1 to about 15 g/day, and administration may continue for a period of at least five days, one week, two weeks, three weeks, one month, two months, three months, six months or 12 months or more.

The exosome-enriched product comprising intact bovine milk-derived exosomes is typically obtained from a whey fraction of bovine milk. In a specific embodiment, the bovine milk-derived exosomes are sourced from a whey-containing bovine milk fraction. By way of example, the whey-containing bovine milk fraction may comprise cheese whey. Generally, the exosomes are obtained from a whey-containing bovine milk fraction using gentle procedures which do not disrupt the exosome vesicle membrane, thereby leaving the exosomes intact and active bioactive agents contained within the exosome structure.

Various methods may be employed to isolate exosomes with care being exercised to avoid disruption of the lipid membrane. Fresh bovine milk, refrigerated bovine milk, thawed frozen bovine milk, or otherwise preserved bovine milk, or any bovine milk fraction containing exosomes, for example, cheese whey, may be employed as a source of exosomes. Isolating the exosomes may comprise performing the isolation immediately upon obtaining milk from a bovine. By way of example, isolating the exosomes may comprise performing the isolation within about 1 day, or about 2 days, or about 3 days, or about 4 days, or about 5 days or about 6 days, or about 7 days from the time of obtaining the milk from a bovine. The exosomes may be isolated within about 10 days, or within about 14 days from the time of obtaining milk from a bovine. Additionally, the bovine milk may be frozen and then thawed for processing for isolating exosomes, with the bovine milk preferably having been frozen within about 1 day, or about 2 days, or about 3 days, or about 4 days, or about 5 days or about 6 days, or about 7 days from the time of obtaining the milk from a bovine. Thawed milk is preferably processed immediately upon thawing. The fresh bovine milk may be subjected to the processing within about 5 days of obtaining the milk from a bovine, or thawed bovine milk which is subjected to processing is thawed from bovine milk that was frozen within about 5 days of obtaining the milk from a bovine.

As mentioned above, a whey-containing bovine milk fraction or, specifically, cheese whey, may serve as a source of exosomes. Cheese whey is the liquid by-product of milk after the formation of curd during the cheese-making or casein manufacturing process. Since cheese whey has already been separated from the casein fraction during the cheese manufacture process, cheese whey has very low casein content. Furthermore, cheese whey advantageously retains more than 50% of milk nutrients, including lactose, fat, proteins, mineral salts, and, surprisingly, a significant number of exosomes that were originally present in the milk in intact form. In addition to these benefits, cheese whey is less expensive than raw milk, and thus using cheese whey as a starting material significantly reduces costs for production of an exosome-enriched product. As such, cheese whey is a novel and promising source for isolating milk exosomes and producing exosome-enriched products.

In a specific embodiment, the cheese whey is obtained by applying an enzyme or enzyme mixture, and more specifically a protease enzyme, for example chymosin, to milk to hydrolyze casein peptide bonds, thus allowing for enzymatic coagulation of casein in the milk. Thus, when the protease enzyme cleaves the protein, it causes the casein in the milk to coagulate and form a gel structure. The casein protein gel network and milk fat then contract together and form curd. The resulting liquid that is separated from the curd is often referred to as sweet whey or cheese whey, typically has a pH from about 6.0 to about 6.5, and comprises whey proteins, lactose, minerals, water, fat and other low level components.

In embodiments of the invention, lean body mass in a pediatric individual is increased during a period of weight gain, thereby promoting healthy catch-up growth. In a specific embodiment, lean body mass is increased during a period of weight gain with the administration of an exosome-enriched product comprising intact bovine milk-derived exosomes in comparison to lean body mass increase during a period of weight gain without the administration of the exosome-enriched product comprising intact bovine milk-derived exosomes.

In another specific embodiment of the invention, an increase in fat mass during a period of weight gain is reduced in a pediatric individual, thereby promoting healthy catch-up growth by reducing fat mass accumulation. In a specific embodiment, an increase in fat mass is reduced during a period of weight gain with the administration of an exosome-enriched product comprising intact bovine milk-derived exosomes in comparison to fat mass increase during a period of weight gain without the administration of the exosome-enriched product comprising intact bovine milk-derived exosomes.

In further embodiments of the invention, a growth rate is increased in a pediatric individual during a period of weight gain, thereby promoting healthy catch-up growth. In a specific embodiment, growth rate is increased during a period of weight gain with the administration of an exosome-enriched product comprising intact bovine milk-derived exosomes in comparison to growth rate increase during a period of weight gain without the administration of the exosome-enriched product comprising intact bovine milk-derived exosomes.

In further embodiments of the invention, weight is increased in a pediatric individual during a period of weight gain, thereby promoting catch-up growth. In a specific embodiment, weight is increased during a period of weight gain with the administration of an exosome-enriched product comprising intact bovine milk-derived exosomes in comparison to weight increase during a period of weight gain without the administration of the exosome-enriched product comprising intact bovine milk-derived exosomes.

In a specific embodiment, the period of weight gain is 4 weeks or 1 month. In yet another embodiment, the period of weight gain is about 1-3 weeks, about 10-16 days, about 7-10 days, about 1-4 weeks, or about 2-3 weeks. In another specific embodiment, the period of weight gain is about 1-4 months, or about 1-6 months, or about 1-12 months, or about 6-12 months. In yet another specific embodiment, the period of weight gain is about 6 months to 18 months, or about 6 months to 24 months, or about 1-2 years, or about 1-5 years.

In another specific embodiment, the individual is a pediatric individual where the pediatric individual is a child at or under the age of about 15 years old, or at or under the age of about 10 years old, or at or under the age of about 5 years old, or at or under the age of about 1 year old, or at or under the age of about 6 months old, or at or under the age of about 3 months old. In a specific embodiment, the pediatric individual is a child at the age of about 5 years old to about 19 years old.

In a specific embodiment, the pediatric individual has or is suffering from at least one of malnourishment, severe weight loss, wasting, under nutrition weight loss, is underweight, has stunted growth, or any combination thereof. In another specific embodiment, the pediatric individual is suffering from malnourishment.

In a specific embodiment, the individual is a malnourished pediatric individual. In another specific embodiment, the individual is a moderately malnourished pediatric individual, or a severely malnourished pediatric individual. In yet another specific embodiment, the moderately malnourished pediatric individual is suffering from one or more of stunting and wasting.

In certain embodiments of the invention, the individual is underweight.

In certain embodiments of the invention, the individual is experiencing a period of severe weight loss.

In a specific embodiment, the exosome-enriched product comprising intact bovine milk-derived exosomes are administered as part of a catch-up diet.

In another specific embodiment, the exosome-enriched product comprising intact bovine milk-derived exosomes are administered in a nutritional composition.

In a further specific embodiment, the exosome-enriched product comprising intact bovine milk-derived exosomes are administered in a nutritional composition as part of a catch-up diet.

The term “nutritional composition” as used herein, unless otherwise specified, refers to nutritional liquids and nutritional powders, the latter of which may be reconstituted or otherwise mixed with a liquid in order to form a nutritional liquid, and are suitable for oral consumption by a human. Nutritional liquids may be prepared in ready-to-drink form or may be reconstituted from powder as described.

In a specific embodiment, the nutritional composition comprising the exosome-enriched product comprising intact bovine milk-derived exosomes is administered orally.

In a specific embodiment, the nutritional composition comprising the exosome-enriched product comprising intact bovine milk-derived exosomes is administered in the form of an exosome-enriched powder. In another specific embodiment, the exosome-enriched product is administered in the form of an exosome-enriched liquid. The exosome enriched product can be administered in either form.

In another specific embodiment, the nutritional composition comprises about 0.001 to about 30 wt %, about 0.001 to about 10 wt %, about 0.001 to about 5 wt %, about 0.001 to about 1 wt %, about 0.01 to about 30 wt %, about 0.01 to about 10 wt %, about 0.01 to about 5 wt %, about 0.01 to about 1 wt %, about 0.1 to about 30 wt %, about 0.1 to about 10 wt %, about 0.1 to about 5 wt %, about 0.1 to about 1 wt %, about 1 to about 30 wt %, about 1 to about 10 wt %, or about 1 to about 5 wt % of the exosome-enriched product comprising the intact bovine milk-derived exosomes, based on the weight of the nutritional composition. In a specific embodiment, the nutritional composition comprises from about 0.001 to about 10 wt % of the exosome-enriched product comprising the intact bovine milk-derived exosomes, based on the weight of the nutritional composition.

In additional embodiments, the nutritional composition is a liquid composition and comprises about 0.001 to about 10 wt %, about 0.001 to about 5 wt %, about 0.001 to about 1 wt %, about 0.01 to about 5 wt %, about 0.01 to about 1 wt %, about 0.1 to about 5 wt %, about 0.1 to about 1 wt %, or about 1 to about 5 wt % of the exosome-enriched product comprising the intact bovine milk-derived exosomes, based on the weight of the liquid nutritional composition. In other embodiments, the nutritional composition is a powdered composition and comprises about 0.01 to about 30 wt %, about 0.01 to about 20 wt %, about 0.01 to about 10 wt %, about 0.01 to about 5 wt %, about 0.1 to about 30 wt %, about 0. 1 to about 20 wt %, about 0.1 to about 10 wt %, about 0.1 to about 5 wt %, about 1 to about 30 wt %, about 1 to about 20 wt %, about 1 to about 10 wt %, or about 1 to about 5 wt % of the exosome-enriched product comprising the intact bovine milk-derived exosomes, based on the weight of the powdered nutritional composition.

In one embodiment of the invention, the nutritional composition comprising an exosome-enriched product comprising intact bovine milk-derived exosomes further comprises one or more of a carbohydrate, a protein and/or a fat. In another embodiment, the nutritional composition comprises carbohydrate, protein and/or fat.

In a specific embodiment, the protein in the nutritional composition comprises whey protein concentrate, whey protein isolate, whey protein hydrolysate, acid casein, sodium caseinate, calcium caseinate, potassium caseinate, casein hydrolysate, milk protein concentrate, organic milk protein concentrate, milk protein isolate, milk protein hydrolysate, nonfat dry milk, condensed skim milk, soy protein concentrate, isolated soy protein, soy protein hydrolysate, pea protein concentrate, pea protein isolate, pea protein hydrolysate, collagen protein, collagen protein isolate, L-Carnitine, taurine, lutein, rice protein concentrate, rice protein isolate, rice protein hydrolysate, fava bean protein concentrate, fava bean protein isolate, fava bean protein hydrolysate, meat proteins, potato proteins, chickpea proteins, canola proteins, mung proteins, guinea proteins, amaranth proteins, chia proteins, hemp proteins, flax seed proteins, earthworm protein, insect protein, one or more amino acids, for example, L-lysine, and/or metabolites thereof, or combinations of two or more thereof. In a specific embodiment, the protein in the nutritional composition comprises milk protein concentrate, isolated soy protein, calcium caseinate, or combinations of two or more thereof. In a further specific embodiment, the protein in the nutritional composition comprises milk protein and/or soy protein.

The one or a mixture of amino acids, which may be described as free amino acids, can be any amino acid known for use in nutritional products. The amino acids may be naturally occurring or synthetic amino acids. In a specific embodiment, the one or more amino acids and/or metabolites thereof comprise one or more branched chain amino acids or metabolites thereof. Examples of branched chain amino acids include arginine, glutamine leucine, isoleucine, and valine. In another specific embodiment, the one or more branched chain amino acids or metabolites thereof comprise alpha-hydroxy-isocaproic acid (HICA, also known as leuic acid), keto isocaproate (KIC), β-hydroxy-β-methylbutyrate (HMB), and combinations of two or more thereof.

The nutritional composition may comprise protein in an amount from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition.

In a specific embodiment, the carbohydrate in the nutritional composition comprises fiber, human milk oligosaccharides (HMOs), maltodextrin, corn maltodextrin, organic corn, corn syrup, sucralose, cellulose gel, cellulose gum, gellan gum, inositol, carrageenan, fructooligosaccharides, hydrolyzed starch, glucose polymers, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arable, sodium carboxymethylcellulose, methylcellulose, guar gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, cornstarch, or combinations of two or more thereof. In a specific embodiment, the nutritional composition comprises one or more carbohydrates comprising sucrose, maltodextrin, cornstarch, fructooligosaccharides, cellulose, or combinations of two or more thereof.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In a specific embodiment, the fat in the nutritional composition comprises coconut oil, fractionated coconut oil, soy oil, soy lecithin, corn oil, safflower oil, high oleic sunflower oil, palm olein, canola oil monoglycerides, lecithin, medium chain triglycerides, one or more fatty acids, including omega-3 fatty acid, such as linoleic acid, alpha-linolenic acid, arachidonic acid, eicosapentaenoic acid, and/or docosahexaenoic acid, olive oil, medium chain triglyceride oil (MCT oil), high gamma linolenic (GLA) safflower oil, palm oil, palm kernel oil, canola oil, marine oils, fish oils, algal oils, borage oil, cottonseed oil, fungal oils, interesterified oils, transesterified oils, structured lipids, or combinations of two or more thereof. In a specific embodiment, the fat in the nutritional composition comprises canola oil, soy oil, medium chain triglyceride oil, or high oleic sunflower oil, or combinations of two or more thereof.

The nutritional composition may comprise fat in an amount of from about 0.5 wt % to about 30 wt % of the nutritional composition. More specifically, the fat may be present in an amount from about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In one embodiment, the nutritional composition is a liquid nutritional composition and comprises from about 1 to about 15 wt % of protein, from about 0.5 to about 10 wt % fat, and from about 5 to about 30 wt % carbohydrate, based on the weight of the nutritional composition.

In another embodiment, the nutritional composition is a powder nutritional composition and comprises from about 10 to about 30 wt % of protein, from about 5 to about 15 wt % fat, and from about 30 wt % to about 65 wt % carbohydrate, based on the weight of the nutritional composition.

In a specific embodiment, the nutritional composition comprises at least one protein comprising milk protein concentrate and/or soy protein isolate, at least one fat comprising canola oil, corn oil, coconut oil and/or marine oil, and at least one carbohydrate comprising maltodextrin, sucrose, and/or short-chain fructooligosaccharide.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth. The nutritional compositions may also include vitamins and minerals, for example in amounts complying with the recommended daily allowances. Non-limiting examples of vitamins include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof. Non-limiting examples of minerals and trace minerals include calcium, phosphorus, magnesium, zinc, manganese, sodium, potassium, molybdenum, chromium, iron, copper, and/or chloride, and combinations thereof.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The intact bovine milk-derived exosomes may be added at any time as desired in the process, for example, to the aqueous solution or to the emulsified blend. The exosomes may be dry blended in powder form with one or more dry ingredients, for example, for combined addition to a liquid composition or if a powdered nutritional product is desirable.

In a specific embodiment, when the nutritional composition is a powder, for example, a serving size is from about 40 g to about 60 g, such as 45 g, or 48.6 g, or 50 g, to be administered as a powder or to be reconstituted in from about 1 ml to about 500 ml of liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

In a specific embodiment, the nutritional composition comprises protein, carbohydrate, fat, and one or more nutrients selected from the group consisting of vitamins, minerals, and trace minerals.

The following Examples demonstrate various embodiments of the invention.

### EXAMPLES

### Example 1: Preparation of Bovine Milk-Derived Exosomes

This example describes a method of isolating bovine milk exosomes from a milk whey fraction, specifically from cheese whey. The method described in this example includes a microfiltration-ultrafiltration isolation method.

An exosome-enriched product containing about 10⁸ to 10¹⁴ intact bovine milk-derived exosomes per gram of the exosome-enriched product was prepared by cascade membrane filtration. First, 1,000 L of sweet cheese whey was processed using tandem multiple ceramic filtration steps. The first microfiltration (MF) step employed a membrane with a molecular weight cut off of 1.4 μm, which yielded a first retentate R1 and a first permeate P1. The first permeate P1 was then subjected to a ultrafiltration (UF) step with a molecular weight cut off of 0.14 μm, which yielded a second retentate R2 and second permeate P2. About 5 volumes of water was added to one volume of the second retentate R2, and the diluted retentate was then passed through the 0.14 μm UF membrane again to remove at least part of the lactose and minerals. The resulting retentate R3 was then combined with an equal volume of water and diafiltered using a 10 kDa membrane to produce a fourth retentate R4. The retentate R4 was diluted with a volume of water five times that of the fourth retentate R4 and diafiltered a second time using the 10 kDa membrane to yield a concentrated retentate, R5. The lactose-free exosome-enriched product R5 was pasteurized at 70° C. for 15 seconds to ensure microbiological stability in order to yield a pasteurized exosome-enriched product R6. The pasteurized exosome-enriched product R6 was subjected to evaporation at about 65° C. to increase the solids content up to 17-18% and then spray-dried at 185° C./85° C. to obtain a exosome-enriched spray-dried product.

### Example 2: Animal Model

This example evaluates the effect of chronic administration of intact bovine milk-derived exosomes on body composition and growth rate using an animal model of weanling male rates of growth retardation or nutritional dwarfing.

The nutritional dwarfing model is based on developing a nutritional stress in weanling male rats placed on restricted intake of a control diet for three weeks (the restriction period). The restricted intake included an intake of 70% of the normal intake amount. The restriction period was followed by another two weeks of a weight gain period to promote catch-up growth (the refeeding period) where the animals had full access to experimental diets.

The restriction period is representative of what a severe period of weight loss would look like in an individual suffering from at least one of malnourishment, wasting, under nutrition weight loss, underweight, growth stunt(s), etc. or any combination thereof.

The refeeding period is representative of what a period would look for an individual undergoing at least one of catch-up growth, a catch-up diet, a period of weight gain, etc. or any combination thereof.

During the restriction period, the animals were divided into two groups. Non-restricted rats (NR group) were fed a rodent standard diet A comprising AIN93G, ad libitum, the composition of which is shown in Table 1, during the entire study period. Restricted rats (RR group) received 70% of the amount of food consumed by the NR group during the restriction period.

After the food restriction period, the NR group was fed a rodent standard diet B comprising AIN93M, shown in Table 1, ad libitum, for the two-week refeeding period. The diet as shown in Table 1 also included other nutritional components such as vitamins, minerals and water that are inherent in the main protein, carbohydrate and fat ingredients. Inherent impurities were also included.

The RR group was divided into two subgroups, TE and EXO, which were fed ad libitum with humanized experimental diets C1 and C2, respectively, for the two-week refeeding period. The composition of diet C1, containing rapidly digestible carbohydrates (RDC blend), is shown in Table 1. Less than 10% of the total energy in the diet was provided by sugars. The EXO group received diet C2, comprising C1, the same base diet as the TE group, but with the addition of an exosome-enriched product comprising intact bovine milk-derived exosomes to the diet (an exosome-enriched diet).

The exosome-enriched product comprising intact bovine milk-derived exosomes that was added consisted of spray dried bovine milk whey exosomes isolated from cheese whey by the microfiltration-ultrafiltration isolation method discussed above in Example 1.

TABLE 1
Composition of Experimental Diets

| | A | B | C1 |
|---|---|---|---|
| Diet | (AIN93G) | (AIN93M) | Base Diet |
| CHO (g/100 g diet) | 63.0 | 71.0 | 66.1 |
| Sucrose (g/100 g CHO) | 14.9 | 13.5 | 6.8 |
| MD 19-24 (g/100 g CHO) | 14.6 | 19.9 | 80.2 |
| Cornstarch (g/100 g) | 63.1 | 59.8 | |
| FOS (g/100 g CHO) | | | 5.7 |
| Cellulose (g/100 g CHO) | 7.4 | 6.8 | |
| Protein (g/100 g diet) | 17.9 | 12.6 | 13.2 |
| Milk Protein | | | 27.4 |
| Concentrate (g/100 g protein) | | | |
| Isolated Soy Protein | | | 19.5 |
| (g/100 g protein) | | | |
| Calcium Caseinate | 100 | 100 | 51.4 |
| (g/100 g protein) | | | |
| Fat (g/100 g diet) | 7.0 | 4.0 | 10.3 |
| Canola Oil (g/100 g fat) | | | 14.5 |
| Soy Oil (g/100 g fat) | 100 | 100 | 42.5 |
| MCT Oil (g/100 g fat) | | | 7.9 |
| High Oleic Sunflower Oil | | | 29.8 |
| (g/100 g fat) | | | |
| Energy (cal/100 g diet) | 394 | 361 | 402 |

During the two-week refeeding period, the weight of each animal was recorded daily and at the end of the refeeding period. Body composition was measured by magnetic resonance imaging (MRI).

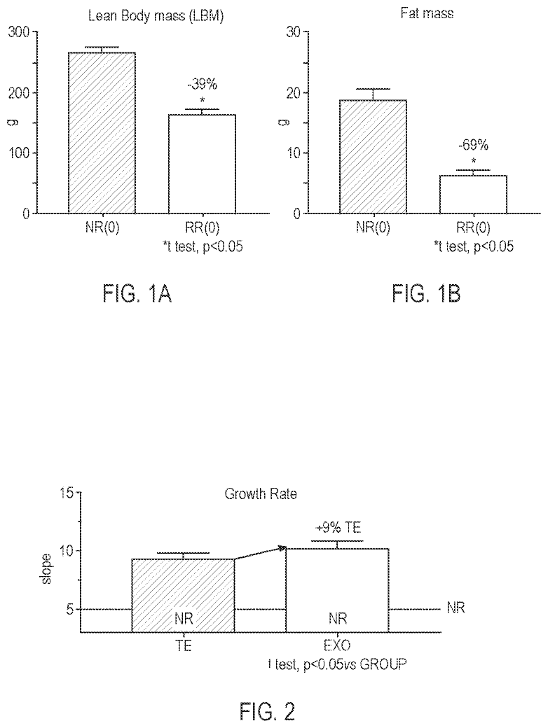

Food deprivation during the restriction period produced a significant deceleration in body weight gain beginning at day two of intervention in the RR group during the restriction period. At the end of three weeks of food deprivation, the RR group had a 44% lower body weight compared to that of the NR group. Similarly, lean body mass and fat mass were also lower in the RR group than in the NR group. Therefore, the three-week restriction period produced a significantly negative effect on growth rate, lean body mass, and fat mass, similar to such characteristics found in wasting children. This is further shown in FIG. 1A which shows lean body mass at the end of the restriction period of both the NR and RR groups, and in FIG. 1B which shows fat mass at the end of the restriction period of both the NR and RR groups.

During the subsequent two-week refeeding period, some of the negative effects on weight from the restriction period were recovered. The degree of recovery varied depending on the composition of the diet administered during the catch-up process (the two-week refeeding period). Both refeeding groups, TE and EXO, showed an elevated growth rate compared to the NR group animals of the same age. However, the EXO group showed about a 9% higher growth rate compared to the TE group during the two-week refeeding period. This is further shown in FIG. 2 comparing the growth rate at the end of the refeeding period between the TE and EXO groups.

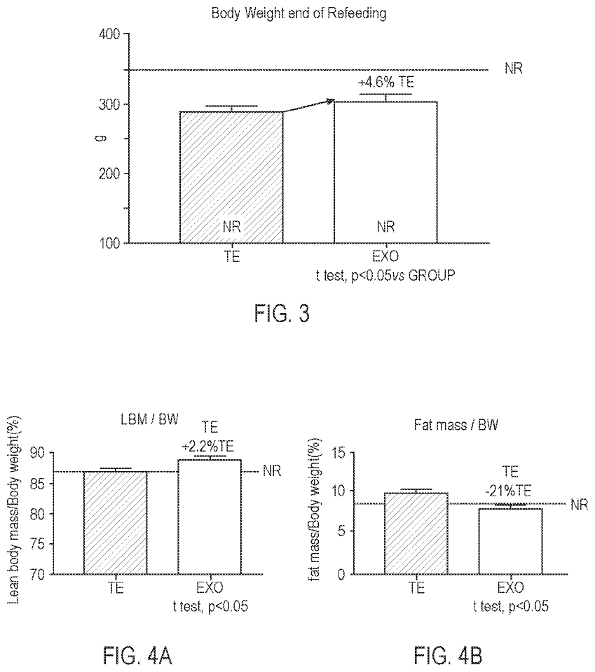

Aside from an increase in growth rate, the EXO group also showed a greater increase in body weight compared to the TE group. As shown in FIG. 3, at the end of the two-week refeeding period, the EXO group animals showed an increase in body weight of about 4.6% greater than that of the TE group.

Additionally, the dietary intervention illustrated in this example showed a recovery in lean body mass and fat mass in both RR groups after refeeding. However, at the end of the refeeding period, the EXO group showed an improvement in lean body mass, with a lower percentage of fat mass compared to the TE group that did not receive bovine milk exosomes. Therefore, the EXO group diet with the addition of the exosome-enriched product comprising intact bovine milk-derived exosomes induced a better recovery of body composition during catch-up growth.

As shown in FIG. 4A, the body composition at the end of the two-week refeeding period shows that the EXO group had a higher increase in lean body mass by about 2.2% compared to the TE group, while also having a lower increase in fat mass by about 21% compared to the TE group, as shown in FIG. 4B. Taken together, these results indicate that a dietary intervention with administration of an exosome-enriched product comprising intact bovine milk-derived exosomes after a period of malnutrition, prevent a catch-up fat phenotype and improve catch-up growth by promoting an increase in lean body mass while maintaining fat mass gain within a normal accretion for healthy growth.

Thus, nutritional supplements or compositions with an exosome-enriched product comprising intact bovine milk-derived exosomes as described herein are advantageous in wasting infants and children, as well as in adolescent and adult subjects, by promoting lean body mass and reducing the development of catch-up fat during catch-up growth. This in turn confers greater protection and resistance against the development of metabolic syndrome, obesity and related diseases throughout life that are associated with increased fat mass.

The specific embodiments and examples described herein are exemplary only and are not limiting to the invention defined by the claims. Additionally, while the present invention has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and processes, or illustrative examples shown and described. Accordingly, departures may be made from such details without departing from the spirit or scope of the general inventive concept.

What is claimed is:

1. A method of promoting healthy catch-up growth in a pediatric individual, comprising administering a nutritional composition comprising an exosome-enriched product comprising intact bovine milk-derived exosomes to the pediatric individual during a period of weight gain.

2. The method of claim 1, wherein the healthy catch-up growth enhances development of lean body mass during the period of weight gain.

3. The method of claim 1 or 2, wherein the healthy catch-up growth reduces development of catch-up fat during the period of weight gain.

4. The method of any one of claims 1-3, wherein the healthy catch-up growth increases a growth rate during the period of weight gain.

5. The method of any one of the preceding claims, wherein the pediatric individual is a child at or under the age of about 15 years old, or at or under the age of about 10 years old, or at or under the age of about 5 years old, or at or under the age of about 1 year old, or at or under the age of about 6 months old, or at or under the age of about 3 months old.

6. The method of any one of the preceding claims, wherein the pediatric individual is a child at the age of about 5 years old to about 19 years old.

7. The method of any one of the preceding claims, wherein the pediatric individual is suffering from a condition comprising at least one of malnourishment, severe weight loss, wasting, under nutrition weight loss, underweight, growth stunt, or any combination thereof.

8. The method of any one of the preceding claims, wherein the pediatric individual is suffering from malnutrition.

9. The method of any one of the preceding claims, wherein the nutritional composition comprising the exosome-enriched product comprising intact bovine milk-derived exosomes further comprises one or more of a carbohydrate, a protein and/or a fat.

10. The method of claim 9, wherein the nutritional composition comprises one or more carbohydrates comprising fiber, human milk oligosaccharides (HMOs), maltodextrin, corn maltodextrin, organic corn, corn syrup, sucralose, cellulose gel, cellulose gum, gellan gum, inositol, carrageenan, fructooligosaccharides, hydrolyzed starch, glucose polymers, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arable, sodium carboxymethylcellulose, methylcellulose, guar gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, cornstarch, or combinations of two or more thereof.

11. The method of claim 9 or 10, wherein the nutritional composition comprises one or more carbohydrates comprising sucrose, maltodextrin, cornstarch, fructooligosaccharides, cellulose, or combinations of two or more thereof.

12. The method of any one of claims 9-11, wherein the nutritional composition comprises one or more proteins comprising whey protein concentrate, whey protein isolate, whey protein hydrolysate, acid casein, sodium caseinate, calcium caseinate, potassium caseinate, casein hydrolysate, milk protein concentrate, organic milk protein concentrate, milk protein isolate, milk protein hydrolysate, nonfat dry milk, condensed skim milk, soy protein concentrate, isolated soy protein, soy protein hydrolysate, pea protein concentrate, pea protein isolate, pea protein hydrolysate, collagen protein, collagen protein isolate, L-Carnitine, taurine, lutein, rice protein concentrate, rice protein isolate, rice protein hydrolysate, fava bean protein concentrate, fava bean protein isolate, fava bean protein hydrolysate, meat proteins, potato proteins, chickpea proteins, canola proteins, mung proteins, guinea proteins, amaranth proteins, chia proteins, hemp proteins, flax seed proteins, earthworm protein, insect protein, one or more amino acids and/or metabolites thereof, or combinations of two or more thereof.

13. The method any one of claims 9-12, wherein the nutritional composition comprises one or more proteins comprising milk protein concentrate, isolated soy protein, calcium caseinate, or combinations of two or more thereof.

14. The method of any one of claims 9-13, wherein the nutritional composition comprises milk protein and/or soy protein.

15. The method of any one of claims 9-14, wherein the nutritional composition comprises one or more fats comprising coconut oil, fractionated coconut oil, soy oil, soy lecithin, corn oil, safflower oil, high oleic sunflower oil, palm olein, canola oil monoglycerides, lecithin, medium chain triglycerides, linoleic acid, alpha-linolenic acid, arachidonic acid, eicosapentaenoic acid, docosahexaenoic acid, olive oil, medium chain triglyceride oil (MCT oil), high gamma linolenic (GLA) safflower oil, palm oil, palm kernel oil, canola oil, marine oils, fish oils, algal oils, borage oil, cottonseed oil, fungal oils, interesterified oils, transesterified oils, structured lipids, or combinations of two or more thereof.

16. The method of any one of claims 9-15, wherein the nutritional composition comprises one or more fats comprising canola oil, soy oil, medium chain triglyceride oil, and high oleic sunflower oil, or combinations of two or more thereof.

17. The method of any one of the preceding claims, wherein the nutritional composition comprising the exosome-enriched product comprising intact bovine milk-derived exosomes is administered to the pediatric individual orally.

18. The method of any one of the preceding claims, wherein the nutritional composition comprising the exosome-enriched product comprising intact bovine milk-derived exosomes is administered as part of a catch-up diet.

19. A method for promoting healthy catch-up growth in an underweight individual, the method comprising administering an exosome-enriched product comprising intact bovine milk-derived exosomes to the individual.

20. The method of claim 19, wherein the individual comprises an individual experiencing a period of severe weight loss.

21. The method of claim 19 or 20, wherein the individual is a moderately malnourished pediatric individual.

22. The method of claim 21, wherein the moderately malnourished pediatric individual suffers from one or more of stunting and wasting.

23. The method of any one of the preceding claims, wherein promoting healthy catch-up growth is achieved by at least one of increasing lean body mass, increasing weight, reducing fat mass accumulation, increasing growth rate, or any combinations thereof during a period of weight gain.

24. The method of any one of the preceding claims, wherein the exosome-enriched product comprising intact bovine milk-derived exosomes is administered chronically during the period of weight gain.
